(19) United States
(12) Patent Application Publication
(10) Pub. No.: US 2025/0186940 A1
(43) Pub. Date: Jun. 12, 2025

(54) MODULAR HUMIDITY MANAGEMENT SYSTEM

(71) Applicant: Honeywell International Inc., Charlotte, NC (US)

(72) Inventors: Rebecca Kamire, Buffalo Grove, IL (US); Emir Rahislic, Chicago, IL (US); Mark Triezenberg, Westmont, IL (US); Bahram Jadidian, Watchung, NJ (US); Jack Ford, Phoenix, AZ (US); Phoebe Henson, Glendale, AZ (US)

(21) Appl. No.: 18/534,091

(22) Filed: Dec. 8, 2023

(51) Int. Cl. B01D 53/22; B01D 53/14; B01D 53/18; B01D 53/26; B60H 3/02; C07C 1/12

(52) U.S. Cl. CPC B01D 53/229; B01D 53/1475; B01D 53/18; B01D 53/268; B60H 3/024; C07C 1/12; B01D 2053/224; B01D 2257/504; B01D 2257/80; B01D 2259/4566; B01D 2259/4575

(57) ABSTRACT

A contaminant removal system includes a humidity management system and a carbon dioxide removal system downstream of the humidity management system. The humidity management system is configured to remove water vapor from a cabin air stream to produce a dehumidified air stream, and includes two or more membrane dehumidifiers. The carbon dioxide removal system is configured to remove carbon dioxide from the dehumidified air stream using a liquid sorbent.

### GOVERNMENT RIGHTS

This invention was made with Government support under Grant Contract Number 80MSFC18C0045 awarded by National Aeronautics and Space Administration (NASA) Marshall Space Flight Center. The Government has certain rights in the invention.

### TECHNICAL FIELD

The present disclosure relates to systems and techniques for removing contaminants from and preserving humidity of an environment.

### BACKGROUND

An environmental control system (ECS) may provide conditioned air to a passenger cabin or other environment. Some of this conditioned air may be treated to remove contaminants, including water vapor. ECS components that are used to remove contaminants may be large and heavy, increasing an overall weight of the ECS, and may consume large amounts of power heating, cooling, and pressurizing various fluid streams. Further, such ECS components may be sized for a high contaminant removal rate, which may not be typical in most operating environments.

### SUMMARY

The disclosure describes systems and techniques for removing contaminants from and managing humidity of cabin air. A contaminant removal system includes a carbon dioxide removal system to remove carbon dioxide from the cabin air through absorption into a liquid sorbent. The liquid sorbent may have a high affinity for water, such that the liquid sorbent may absorb a portion of the humidity in the cabin air. An efficiency of carbon dioxide removal and the reliability of components of the carbon dioxide removal system may be related to a concentration of water in the liquid sorbent, as a higher concentration of water in the liquid sorbent may increase an amount of power consumed by the carbon dioxide removal system, increase a load on a vacuum source used to desorb the carbon dioxide, and reduce a reliability of the vacuum source due to condensation of the water at the vacuum source.

To avoid too much humidity being removed from the cabin air, the contaminant removal system includes a humidity management system upstream of the carbon dioxide removal system to control the humidity in the cabin air prior to supplying the cabin air to the carbon dioxide removal system. The humidity management system removes water vapor from the cabin air prior to removing carbon dioxide and adds at least a portion of the water vapor back into a decontaminated air stream. A conventional carbon dioxide removal system may not remove any humidity from a cabin. Instead, humidity is removed by a condensing heat exchanger. However, the humidity in the cabin air may be variable due to various factors in the cabin, such as a presence and number of personnel in the cabin and an activity level of the personnel.

Separate generation and removal results in humidity and carbon dioxide concentrations varying somewhat independently, such that the variation in humidity may not track a variation in carbon dioxide or other contaminants. For example, during exercise by each member of a crew, the carbon dioxide generation rate may substantially increase, such as by double, while humidity generation rate may increase even more, such as by quadruple. During sleep, both generation rates for carbon dioxide and humidity decrease somewhat. Additionally, ranges of carbon dioxide and humidity may be substantially different, such that even generation rates that do track may be subject to control based on different limits being reached. For example, humidity in the cabin can vary substantially day to day, but carbon dioxide concentrations must remain in a very tight range for crew health and comfort. As such, configuring a humidity management system based on a highest anticipated humidity may result in too much or too little humidity being removed for a target concentration of carbon dioxide.

Contaminant removal systems described herein include humidity management systems configured to vary the removal rate of humidity from the cabin air. The humidity management system includes multiple membrane dehumidifiers that can be modularly selected and arranged based on a desired removal rate of humidity from the cabin air. For example, a manifold system may be configured to select a particular number of membrane dehumidifiers to handle a particular volume of cabin air and/or humidity in the cabin air, and/or may arrange the membrane dehumidifiers, such as in parallel or series, to have a particular capacity. As a result, the contaminant removal systems may be capable of varying the humidity removal rate for a particular flow rate of cabin air independently of the carbon dioxide removal rate.

In some examples, the disclosure describes a contaminant removal system that includes a humidity management system and a carbon dioxide removal system downstream of the humidity management system. The humidity management system is configured to remove water vapor from a cabin air stream to produce a dehumidified air stream, and includes two or more membrane dehumidifiers. The carbon dioxide removal system is configured to remove carbon dioxide from the dehumidified air stream using a liquid sorbent.

In some examples, the disclosure describes a method for removing contaminants from an environment. The method includes removing, by a humidity management system, water vapor from a cabin air stream to produce a dehumidified air stream, in which the humidity management system includes two or more membrane dehumidifiers, and removing, by a carbon dioxide removal system, carbon dioxide from the dehumidified air stream using a liquid sorbent.

### BRIEF DESCRIPTION OF THE FIGURES

The details of one or more examples are set forth in the accompanying drawings and the description below. Other features, objects, and advantages will be apparent from the description and drawings, and from the claims.

FIG. 1A is a block diagram illustrating an example contaminant removal system for removing contaminants from and managing a humidity of a cabin air stream.

FIG. 1B is a flowchart of an example method for removing contaminants from and managing a humidity of a cabin air stream.

FIG. 2 is a schematic diagram illustrating an example humidity management system for managing a humidity of a cabin air stream.

FIG. 3 is a schematic diagram illustrating an example contaminant removal system for removing contaminants from and managing a humidity of a cabin air stream.

FIGS. 4A-4C are graphs illustrating removal of carbon dioxide from a cabin air stream.

FIGS. 5A-5C are graphs illustrating removal of water vapor from a cabin air stream.

### DETAILED DESCRIPTION

The disclosure describes systems and techniques for removing contaminants from and managing humidity of cabin air. A contaminant removal system includes a carbon dioxide removal system that removes carbon dioxide using a liquid sorbent and a humidity management system that removes water vapor using membrane dehumidifiers that can be modularly selected and arranged for varied humidity removal rates. Contaminant removal systems described herein may be utilized as part of an environmental control system (ECS), such as in spacecraft, aircraft, watercraft, and the like. In some examples, contaminant removal systems may be used in an ECS of a resource-limited environment, such as a passenger cabin of a spacecraft, in which carbon dioxide and water may be recycled to produce oxygen gas, water, methane, hydrogen gas, and a variety of other compounds used in life support systems. Such resource-limited environments may be particularly suited for a contaminant removal system that includes components that may be selectively operated for more efficient humidity management.

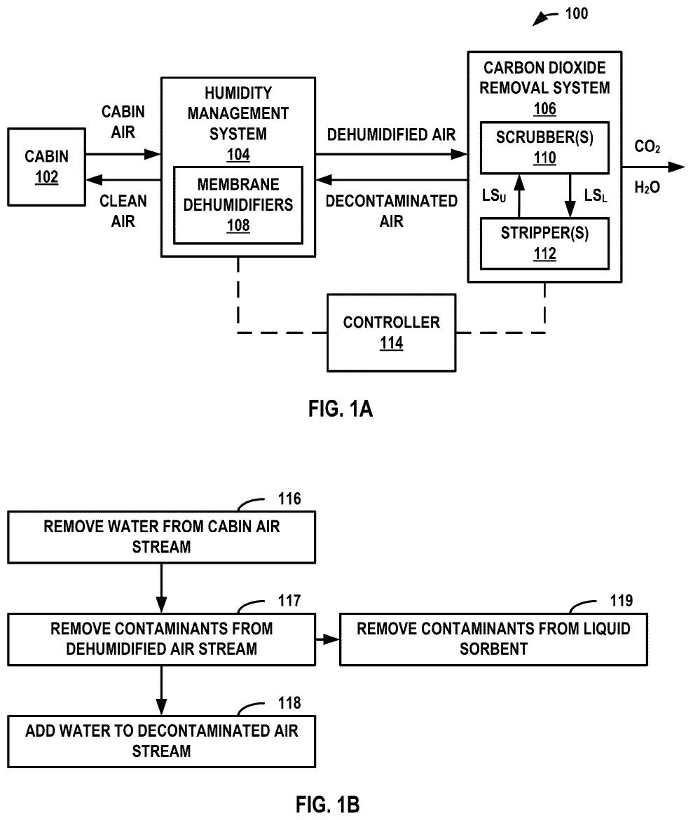

FIG. 1A is a block diagram illustrating an example contaminant removal system 100 for removing contaminants from and managing a humidity of a cabin air stream. Contaminant removal system 100 is configured to remove contaminants from a cabin 102. Cabin 102 may be a controlled environment, such as an aircraft cabin, spacecraft cabin, watercraft cabin, or the like, and contaminants removed from cabin 102 may include, but are not limited to, carbon dioxide, water, hydrocarbons, permanent gases, or the like. In the example of FIG. 1A, cabin 102 is a cabin of a closed-loop system, such as a spacecraft cabin or submarine cabin, in which components of a cabin air stream from cabin 102, such as carbon dioxide and water, may be removed within contaminant removal system 100, allowing a purified supply air stream to be generated and carbon dioxide and water to be recovered. However, in other examples, cabin 102 may be a cabin of an open-loop system, such as an aircraft cabin, in which components of a cabin air stream may be removed to generate a purified supply air stream with only partial or no subsequent recovery of the contaminants.

System 100 includes a carbon dioxide removal system 106 configured to remove one or more contaminants using a liquid sorbent. A liquid sorbent may include any liquid configured to absorb and desorb a gaseous species. Liquid sorbents may be water soluble, hygroscopic (i.e., capable of absorbing moisture from the air), capable of absorbing or desorbing contaminants in response to a change in solubility driven by a change in temperature, and/or capable of releasing water by evaporation, such as by elevating the temperature or reducing the water partial pressure. In some examples, the liquid sorbent may be an ionic liquid sorbent. Ionic liquid sorbents may be salts that are generally comprised of an anion and an organic cation. These salts may be liquid at their temperature of use, have effectively zero vapor pressure, be generally nontoxic, and/or have sufficient stability to resist deterioration. In some examples, ionic liquid sorbents may contain relatively large organic cations and any of a variety of anions, which may be tailored to obtain desired characteristics, such as characteristics that improve absorption of the particular contaminant under operating conditions of carbon dioxide removal system 106. The liquid sorbent may be selected for a variety of properties related to contact with a hydrophobic membrane and absorption of carbon dioxide including, but not limited to, a high capacity for carbon dioxide, a low viscosity, and a high stability. A variety of ionic liquid sorbents may be used including, but not limited to, imidazolium salts, such as 1-ethyl-3-methylimidazolium (EMIM) acetate (Ac).

Liquid sorbents may be used with membrane contactors, such as scrubbers, that contact an air stream with the liquid sorbent across one or more hydrophobic porous membranes. Absorption of the contaminants by the liquid sorbent may be determined by a concentration of the contaminants in the corresponding air stream. In general, a concentration of water in an air stream from human occupied environments may be substantially higher than a concentration of carbon dioxide or other gaseous contaminants. As a result, absorption of water vapor from the air stream into the liquid sorbent is higher than absorption of carbon dioxide from the air stream.

Liquid sorbents may be present in a liquid sorbent mixture with other components. A capacity of the liquid sorbent for a particular contaminant may be affected by a concentration of the liquid sorbent in the liquid sorbent mixture. For example, as a concentration of the liquid sorbent in the liquid sorbent mixture decreases, an amount of carbon dioxide that may be absorbed by the liquid sorbent may decrease. Further, a higher amount of water in the liquid sorbent mixture may increase an amount of power required to remove the water through evaporation or reduce a service life of components, such as a vacuum pump, that generate a vacuum on the membrane contactor.

System 100 includes humidity management system 104 configured to remove a portion of water vapor or other gaseous contaminants in a cabin air stream prior to removal of carbon dioxide and subsequently add a portion of the removed water to decontaminated air supplied to cabin 102 to maintain the humidity of cabin 102 and/or maintain a water concentration in carbon dioxide removal system 106. Humidity management system 104 is configured to remove water from the cabin air stream using two or more membrane dehumidifiers 108. Each membrane dehumidifier is configured to transfer humidity from a relatively humid cabin air stream from cabin 102 across one or more membranes to a relatively dry decontaminated air stream from carbon dioxide removal system 106. By removing at least a portion of the water in the cabin air stream prior to carbon dioxide removal, components of carbon dioxide removal system 106 may remove carbon dioxide more efficiently and/or reliably.

Carbon dioxide removal system 106 includes at least one scrubber 110 and at least one stripper 112. Scrubber 110 is configured to absorb carbon dioxide from the dehumidified air stream into the liquid sorbent and discharge a decontaminated air stream having a lower concentration of carbon dioxide than the dehumidified air stream to humidity management system 104 for humidification. Stripper 112 is configured to desorb carbon dioxide, and optionally other contaminants, from the liquid sorbent into a contaminant stream for discharge, storage, or further processing. A liquid sorbent loop circulates a loaded liquid sorbent (LS_{L}) stream from scrubber 110 to stripper 112 and an unloaded liquid sorbent (LS_{U}) stream from stripper 112 to scrubber 110. Removal of water from the cabin air stream by humidity management system 104 may limit an amount of water that is absorbed into the liquid sorbent. The liquid sorbent may absorb other gaseous contaminants, such as hydrocarbons, that may not be removed from cabin air stream 116 by humidity management system 104.

Contaminant removal system 100 may include a process control system that includes a controller 114 and one or more sensor sets (not shown). Controller 114 may include any of a wide range of devices, including control circuitry, processors (e.g., one or more microprocessors, one or more application specific integrated circuits (ASICs), one or more field programmable gate arrays (FPGAs), or the like), processing circuitry, one or more servers, one or more desktop computers, one or more notebook (i.e., laptop) computers, one or more cloud computing clusters, or the like.

Controller 114 may be communicatively coupled to and configured to receive measurement signals from the one or more sensor sets, and other process control components (not shown) of contaminant removal system 100, such as: control valves for various streams; pumps; heaters; heat exchangers; compressors; and the like. The sensor sets may include instrumentation configured to detect any of a pressure, temperature, flow rate, and/or contaminant concentration (e.g., carbon dioxide concentration or water concentration) of a liquid or gas stream of contaminant removal system 100. Controller 114 may be configured to use the detected conditions to control operation of contaminant removal system 100 to function as described in the application.

Controller 114 is configured to control conditions within membrane dehumidifiers 108, scrubber 110, and/or stripper 112. For example, as will be described further below, controller 114 may be configured to control conditions at membrane dehumidifiers 108 to control a humidity of the dehumidified air stream to prevent water losses at stripper 112; control a humidity of the dehumidified air stream and the rehumidified air stream to maintain a humidity in cabin 102; control conditions at scrubber 110 and stripper 112 to control a concentration of carbon dioxide in cabin 102 (e.g., below a threshold) and a concentration or flow rate of carbon dioxide in the contaminant stream. Scrubber 110 and stripper 112 are intimately related, such that conditions at both scrubber 110 and stripper 112 will impact the concentration of carbon dioxide scrubbed and stripped, concentration of carbon dioxide in the cabin, and concentration of carbon dioxide in the product contaminant stream.

To maintain a safe and/or comfortable environment in cabin 102, controller 114 is configured to control a concentration of carbon dioxide within the environment of cabin 102. For example, controller 114 may be configured to receive a concentration measurement for carbon dioxide, such as from a cabin air sensor set or a carbon dioxide concentration sensor in cabin 102, and determine whether the concentration measurement of carbon dioxide exceeds a concentration setpoint. For example, the concentration setpoint may be a threshold contaminant concentration. Controller 114 may be configured to send, in response to the concentration measurement of carbon dioxide exceeding the concentration setpoint, a control signal to decrease a concentration of carbon dioxide in the rehumidified air stream discharged by scrubber 110. For example, controller 114 may send a control signal to control a flow rate of the liquid sorbent mixture between scrubber 110 and stripper 112; a flow rate, humidity, and/or temperature of a sweep gas stream (not shown) into stripper 112; a temperature of the liquid sorbent mixture at scrubber 110 or stripper 112; a flow rate of the cabin air stream; or any other variable that may control a rate of removal of carbon dioxide from the cabin air stream.

In addition to controlling a concentration of carbon dioxide, controller 114 is configured to control a concentration of water (or humidity) in the dehumidified air stream discharged to scrubber 110. For example, controller 114 may be configured to receive a humidity measurement, such as from a humidity sensor in the dehumidified air stream, and determine whether the humidity measurement exceeds a maximum humidity setpoint for scrubber 110. For example, the humidity setpoint may be a target humidity of the dehumidified air stream for maintaining a desired water content of the liquid sorbent mixture. Controller 114 may be configured to send, in response to the humidity measurement being above a maximum humidity setpoint, a control signal to change a humidity in the rehumidified air stream. For example, controller 114 may send a control signal to control selection of one or more of membrane dehumidifiers 108; a flow rate of the cabin air stream; or any other variable that may control a rate of removal of water vapor from the cabin air stream.

In some examples, controller 114 is configured to control a concentration of water in the rehumidified air stream discharged to cabin 102. For example, controller 114 may be configured to receive a humidity measurement, such as from a cabin air sensor set or a humidity concentration sensor in cabin 102, and determine whether the humidity measurement exceeds a maximum or minimum humidity setpoint. For example, the humidity setpoint may be a target humidity of the rehumidified air stream for maintaining cabin 102 within a humidity range, such as a target humidity range for passenger comfort between about 5% and about 75% relative humidity. Controller 114 may be configured to send, in response to the humidity measurement being outside a humidity range setpoint, a control signal to change a humidity in the rehumidified air stream. For example, controller 114 may send a control signal to control selection of one or more of membrane dehumidifiers 108; a flow rate of the cabin air stream; or any other variable that may control a rate of removal of water vapor from the cabin air stream and addition of the water vapor to the decontaminated air stream.

In some examples, controller 114 is configured to control a water content of the liquid sorbent in the liquid sorbent loop of carbon dioxide removal system 106. For example, a portion of water in the liquid sorbent mixture may be desorbed at stripper 112 into the contaminant stream. Without replacement of the desorbed water, a viscosity of the liquid sorbent mixture may increase, thereby reducing mass transfer of carbon dioxide into the liquid sorbent. To replace the desorbed water, controller 114 may be configured to determine whether a concentration of water, or a related parameter such as concentration of liquid sorbent, in the liquid sorbent mixture is outside a concentration range setpoint. For example, the concentration range setpoint may be the target concentration range of water in the liquid sorbent mixture in the liquid sorbent loop for efficiently absorbing and desorbing carbon dioxide while maintaining a sufficiently low viscosity. Controller 114 may be configured to receive a concentration measurement, such as from a concentration sensor fluidically coupled to the dehumidified air stream, the loaded liquid sorbent stream, or the unloaded liquid sorbent stream, and send, in response to the concentration measurement being outside the concentration range setpoint, a control signal to change the humidity of the dehumidified air stream. For example, controller 114 may send a control signal to control a number or configuration of membrane dehumidifiers 108; a flow rate of the cabin air stream; or any other variable that may control a rate of removal of water vapor from the cabin air stream.

FIG. 1B is a flowchart of an example method for removing contaminants from and maintaining a humidity of a cabin air stream, and will be described with respect to contaminant removal system 100 of FIG. 1A. The example of FIG. 1B includes removing, by humidity management system 104, water from a cabin air stream (116). For example, controller 114 may detect that the cabin air stream has a concentration of one or more contaminants that is above a threshold and, in response, control components of the cabin air circuit to control a flow rate of cabin air stream 116. Controller 114 may control removal of water vapor based on this flow rate of the cabin air stream. Removing water from the cabin air stream may include transferring, by one or more membrane dehumidifiers 108, a portion of water vapor from the cabin air stream to the decontaminated air stream. For example, controller 114 may control components of humidity management system 104 to selectively couple one or more membrane dehumidifiers 108 to achieve a desired water removal rate. After removal, humidity management system 104 may discharge the dehumidified air stream to carbon dioxide removal system 106. A concentration of water vapor in the dehumidified air stream may be lower than a concentration of water vapor in the cabin air stream. In some examples, a concentration of water in the dehumidified air stream may be further controlled to maintain a desired water content of the liquid sorbent mixture in the liquid sorbent loop of carbon dioxide removal system 106.

The example of FIG. 1B includes removing, by carbon dioxide removal system 106, contaminants, including carbon dioxide, from the dehumidified air stream (117). Removing contaminants from the dehumidified air stream may include absorbing, by scrubber 110, a portion of carbon dioxide from the dehumidified air stream into the liquid sorbent. For example, controller 114 may control components of the liquid sorbent loop of carbon dioxide removal system 106 to control a flow rate of the liquid sorbent between scrubber 110 and stripper 112 and a temperature of the liquid sorbent prior to entry into scrubber 110. After removal of the contaminants, carbon dioxide removal system 106 discharges the decontaminated air stream back to humidity management system 104.

As part of the contaminant removal function, the example of FIG. 1B includes removing, by carbon dioxide removal system 106, contaminants from the liquid sorbent (119). Removing the contaminants may include desorbing, by stripper 112, the carbon dioxide and other contaminants from the liquid sorbent to produce a contaminant stream. For example, controller 114 may control components of the liquid sorbent loop of carbon dioxide removal system 106 to control a flow rate of the liquid sorbent between scrubber 110 and stripper 112 and a temperature of the liquid sorbent prior to entry into stripper 112. The contaminant stream may be stored or further processed, such to produce hydrocarbons using a Sabatier reactor.

The example of FIG. 1B includes adding, by humidity management system 104, water to the decontaminated air stream (118). Adding water to the decontaminated air stream may include transferring, by one or more membrane dehumidifiers 108, a portion of the water from the cabin air stream to the decontaminated air stream to produce a rehumidified air stream. For example, controller 114 may control components of humidity management system 104 to selectively couple one or more membrane dehumidifiers 108 to achieve a desired water removal rate. While illustrated in FIG. 1B as being separate steps, removing water from the cabin air stream and adding water to the decontaminated air stream may occur as one step (e.g., transfer of water vapor through membranes of membrane dehumidifiers 108). After the decontaminated air stream has been rehumidified, humidity management system 104 may discharge the rehumidified air stream back to cabin 102.

Humidity management system 104 may enable contaminant removal system 100 to modularly control humidity in an air stream using a variable humidity rate that is independent of a carbon dioxide removal rate of carbon dioxide removal system 106. For example, a contaminant removal system may operate based on a flow rate of a cabin air stream that corresponds to a carbon dioxide removal rate to maintain a concentration of carbon dioxide within a limit. A contaminant removal system that includes a set number of membrane dehumidifiers may be sized for a maximum anticipated flow rate of a cabin air stream and a maximum anticipated concentration of humidity in the cabin air stream. As a result, the contaminant removal system may have a set capacity that may remove too much water at low flow rates of the cabin air stream and too little water at high flow rates of the cabin air stream. In contrast, humidity management system 104 may select a different number or configuration of membrane dehumidifiers 108 to provide a desired water vapor removal rate.

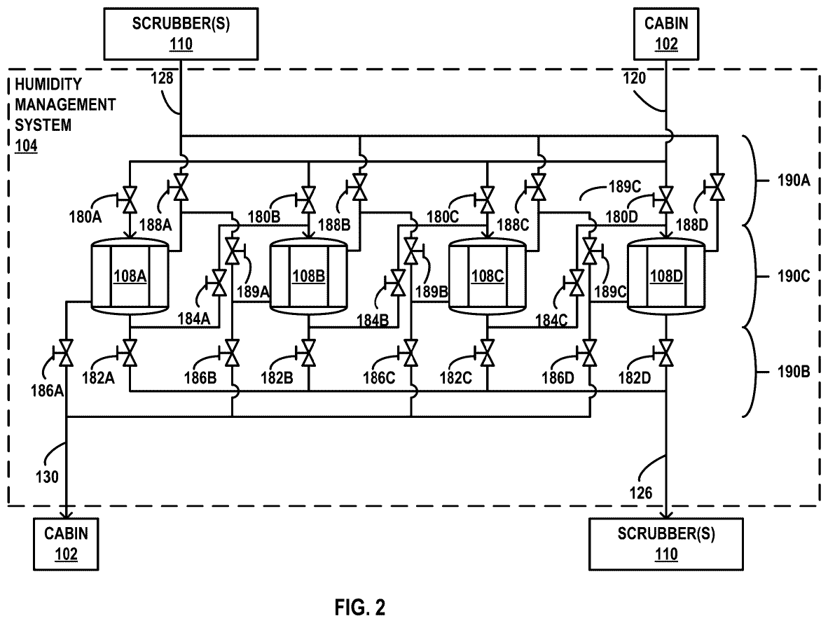

FIG. 2 is a more detailed schematic diagram illustrating the example humidity management system 104 of FIG. 1A for managing a humidity of a cabin air stream 120 from cabin 102. In the example of FIG. 2, humidity management system 104 is configured to receive cabin air stream 120 from cabin 102, remove humidity from the cabin air, and discharge a dehumidified air stream to scrubber 110. Humidity management system 104 is also configured to receive a decontaminated air stream 128 from scrubber 110, add humidity to the decontaminated air, and discharge a rehumidified air stream 130 back to cabin 102.

Humidity management system 104 includes a plurality of membrane dehumidifiers 180. In the example of FIG. 2, humidity management system 104 includes four membrane dehumidifiers 108A, 108B, 108C, 108D; however, in other examples, humidity management system 104 may include other numbers of membrane dehumidifiers 108. A number of membrane dehumidifiers 108 in humidity management system 104 may be based on a number of factors including, but not limited to, a redundancy of membrane dehumidifiers 108, an operating range of membrane dehumidifiers 108, and other factors that may be related to a water removal capacity during operation. Housings of membrane dehumidifiers 108A-D may be manifolded, instead of existing as stand-alone dehumidifiers, with all four inlet and outlet ports. Combining a housing may save mass and volume versus the corresponding stand-alone dehumidifiers, and minimize the size, weight, and volume impact of going from a single large dehumidifier to a modular approach.

Each membrane dehumidifier 108 may have a removal rate for transferring contaminants from one gas stream to another gas stream, and includes one or more membranes that have a particular surface area. The removal rate of a particular membrane dehumidifier may be related to a surface area of the membranes in the membrane dehumidifier 108. In some examples, each membrane dehumidifier 108 has a similar removal rate. For example, membrane dehumidifiers 108 configured for parallel operation may have a similar removal rate to maintain a substantially similar pressure drop across each membrane dehumidifier 108. In other examples, at least two membrane dehumidifiers 108 have different removal rates. The different removal rates may be tiered based on different operating levels within an operating envelope, different operating modes, environmental conditions in cabin 102, or other variations for which a change in removal rate may not be linear. For example, a first membrane dehumidifier 108A may have a removal rate for a lowest removal rate of water vapor anticipated for an operating envelope of carbon dioxide removal system 106. Other membrane dehumidifiers 108B, 108C, 108D may each have a smaller removal rate than first membrane dehumidifier 108A that increments to a removal rate for a highest removal rate of water vapor anticipated for the operating envelope of carbon dioxide removal system 106.

In the example of FIG. 2, humidity management system 104 includes a manifold system 190. Manifold system 190 is configured to selectively couple each membrane dehumidifier 108 to control flow of air streams to and from each membrane dehumidifier 108. Manifold system 190 includes an inlet manifold subsystem 190A, an outlet manifold subsystem 190B, and an interconnect manifold subsystem 190C. Together, inlet manifold subsystem 190A and outlet manifold subsystem 190B may be configured to fluidically couple each membrane dehumidifier 108 to cabin air stream 120, dehumidified air stream 126, decontaminated air stream 128, and rehumidified air stream 130.

Inlet manifold subsystem 190A is configured to control flow of cabin air from cabin air stream 120 to a respective membrane dehumidifier 108. For example, inlet manifold subsystem 190A includes an inlet valve 180A, 180B, 180C, 180D configured to control flow of cabin air from cabin air stream 120 to a respective membrane dehumidifier 108A, 108B, 108C, 108D. Outlet manifold subsystem 190B is configured to control flow of dehumidified air from a respective dehumidifier 108 to dehumidified air stream 126. For example, outlet manifold subsystem 190B includes outlet valves 182A, 182B, 182C, 182D configured to control flow of dehumidified air from a respective membrane dehumidifier 108A, 108B, 108C, 108D to dehumidified air stream 126.

In addition to controlling flow of cabin air and dehumidified air, inlet manifold subsystem 190A and/or outlet manifold subsystem 190B may be configured to control flow of decontaminated air from decontaminated air stream 128 to a respective membrane dehumidifier 108 and/or control flow of rehumidified air from a respective membrane dehumidifier 108 to rehumidified air stream 130. For example, outlet manifold subsystem 190B inlet manifold subsystem 190B includes an inlet valve 188A, 188B, 188C, 188D configured to control flow of decontaminated air from decontaminated air stream 128 to a respective membrane dehumidifier 108A, 108B, 108C, 108D, and outlet manifold subsystem 190B includes an outlet valve 186A, 186B, 186C, 186D configured to control flow through a respective membrane dehumidifier 108A, 108B, 108C, 108D, thereby controlling flow of both dehumidifier air into and rehumidified air from the respective membrane dehumidifier 108A, 108B, 108C, 108D. However, in other examples, inlet manifold subsystem 190A may control fluidic coupling of the respective membrane dehumidifiers 108.

In the example of FIG. 2, manifold subsystems 190A-C may be further configured to fluidically couple one or more membrane dehumidifiers 108 to another membrane dehumidifier 108. For example, interconnect manifold subsystem 190C may be configured to fluidically couple an outlet of membrane dehumidifier 108A to an inlet of membrane dehumidifier 108B via interconnect valve 184A, an outlet of membrane dehumidifier 108B to an inlet of membrane dehumidifier 108C via interconnect valve 184B, and an outlet of membrane dehumidifier 108C to an inlet of membrane dehumidifier 108D via interconnect valve 184C. As another example, interconnect manifold subsystem 190C may be configured to fluidically couple an outlet of membrane dehumidifier 108A to an inlet of membrane dehumidifier 108B via interconnect valve 189A, an outlet of membrane dehumidifier 108B to an inlet of membrane dehumidifier 108C via interconnect valve 189B, and an outlet of membrane dehumidifier 108C to an inlet of membrane dehumidifier 108D via interconnect valve 189C.

In some examples, manifold system 190 may be configured to fluidically couple membrane dehumidifiers 108A-D in parallel with respect to the cabin air stream 120 and/or decontaminated air stream 128. For example, to connect membrane dehumidifiers 108A and 108B in parallel, inlet manifold subsystem 190A may open inlet valves 180A and 180B to fluidically couple membrane dehumidifiers 108A and 108B to cabin air stream 120, outlet manifold subsystem 190B may open outlet valve 182A and 182B and outlet valves 186A and 186 to fluidically couple membrane dehumidifiers 108A and 108B to dehumidified air stream 126, decontaminated air stream 128, and rehumidified air stream 130, and interconnect manifold subsystem 190C may close all interconnect valves 184A-C. As a desired removal rate of humidity changes, manifold system 190 may fluidically couple or decouple one or more additional membrane dehumidifiers 108 to increase a residence time of cabin air in each membrane dehumidifier 108 and/or decrease a pressure drop across each membrane dehumidifier 108. Membrane dehumidifiers 108A-D fluidically coupled in parallel may have lower pressure drop, and correspondingly lower power required, and lower air velocity, and correspondingly higher efficiency, compared to membrane dehumidifiers fluidically coupled in series.

In some examples, manifold system 190 may be configured to fluidically couple membrane dehumidifiers 108A-D in series with respect to cabin air stream 120 and/or decontaminated air stream 128. For example, to connect membrane dehumidifiers 108A and 108B in series, inlet manifold subsystem 190A may open inlet valve 180A to fluidically couple membrane dehumidifier 108A to cabin air stream 120 and shut inlet valve 180B, outlet manifold subsystem 190B may both open outlet valve 182B to fluidically couple membrane dehumidifier 108B to dehumidified air stream 126, and open outlet valves 186A and 186B to fluidically couple membrane dehumidifiers 108A and 108B to decontaminated air stream 128 and rehumidified air stream 130, and interconnect manifold subsystem 190C may open interconnect valve 184A to fluidically couple an outlet of membrane dehumidifier 108A to an inlet of membrane dehumidifier 108B.

In some examples, manifold system 190 may be configured to fluidically couple membrane dehumidifiers 108A-D in both parallel and series with respect to cabin air stream 120 and/or decontaminated air stream. For example, inlet manifold subsystem 190A may open inlet valves 182A and 182C to fluidically couple membrane dehumidifiers 108A and 108C to cabin air stream 120 and shut inlet valves 182B and 182D, outlet manifold subsystem 190B may open outlet valves 182B and 182D to fluidically couple membrane dehumidifiers 108B and 108D to dehumidified air stream 126 and shut outlet valves 182A and 182C, outlet manifold subsystem 190B may open all outlet valves 186A-186D to fluidically couple membrane dehumidifiers 108A-108D to decontaminated air stream 128 and rehumidified air stream 130, and interconnect manifold subsystem 190C may open interconnect valves 184A and 184C and shut 184B to fluidically couple an outlet of membrane dehumidifier 108A to an inlet of membrane dehumidifier 108B, and an outlet of membrane dehumidifier 108C to an inlet of membrane dehumidifier 108D.

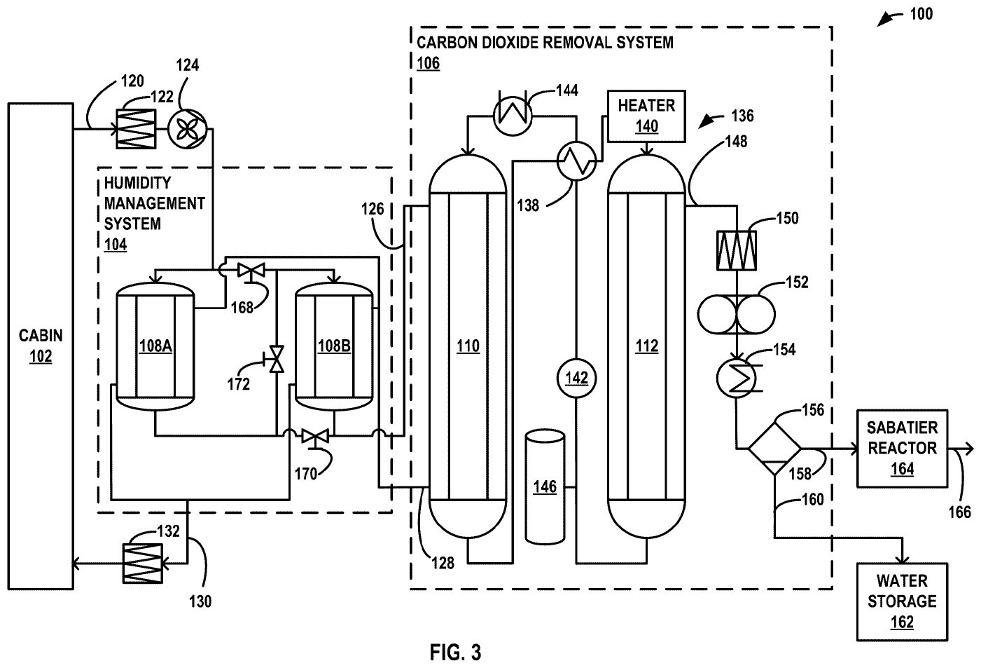

FIG. 3 is a more detailed schematic diagram illustrating the example contaminant removal system 100 of FIG. 1A for removing contaminants from and maintaining a humidity of a cabin air stream 120. In the example of FIG. 3, cabin 102 may be a cabin of a closed-loop system, such as a spacecraft cabin or submarine cabin, in which components of cabin air stream 120 from cabin 102, such as carbon dioxide and water, may be removed within contaminant removal system 100, allowing a purified rehumidified air stream 130 to be generated. In some examples, cabin air stream 120 may have a carbon dioxide concentration between about 1000 ppm and about 5000 ppm and/or a hydrocarbon concentration less than about 100 ppm. Rehumidified air stream 130 has a lower concentration of carbon dioxide than cabin air stream 120. For example, rehumidified air stream 130 may have a concentration of carbon dioxide that is about 25% to about 99% less than a concentration of carbon dioxide in cabin air stream 120, such as about 40% to about 95% less than the concentration of carbon dioxide in cabin air stream 120, or such as about 60% to about 80% less than the concentration of carbon dioxide in cabin air stream 120.

Contaminant removal system 100 includes a cabin air circuit (not labeled) configured to circulate cabin air between cabin 102, humidity management system 104, and carbon dioxide removal system 106. In the example of FIG. 3, cabin air stream 120 includes a filter 122 configured to remove particulates from cabin air stream 120 prior to entry into membrane dehumidifiers 108 and a blower 124 configured to draw cabin air into membrane dehumidifiers 108, while rehumidified air stream 130 includes a filter 132 configured to remove any leaked liquid sorbent and/or further filter clean air from rehumidified air stream 130 prior to entry into cabin 102.

Humidity management system 104 includes two or more membrane dehumidifiers 108. In the example of FIG. 3, humidity management system 104 includes a first membrane dehumidifier 108A and a second membrane dehumidifier 108B. However, humidity management system 104 may include any plurality of membrane dehumidifiers 108. In some examples, humidity management system 104 may include a number of membrane dehumidifiers 108 corresponding to a water vapor removal rate for removing water vapor at a maximum anticipated flow rate of cabin air stream 120 and a maximum anticipated water vapor generation rate in cabin 102. Humidity management system 104 includes valves 168, 170, 172 configured to select between one or both membrane dehumidifiers 108A and 108B and arrange membrane dehumidifiers 108A and 108B between parallel and series operation, such as described in FIG. 2.

Each membrane dehumidifier 108 is configured to return humidity from cabin air stream 120 to decontaminated air stream 128 and discharge a dehumidified air stream 126 to scrubber 110. On one side, each dehumidifier 108 is configured to receive cabin air stream 120 as a feed gas stream and discharge dehumidified air stream 126 to scrubber 110 having a lower humidity. As a result, dehumidified air from dehumidified air stream 126 discharged from dehumidifiers 108 may have a lower humidity than cabin air from cabin air stream 120 received by dehumidifiers 108. For example, dehumidified air stream 126 may have a humidity that is between about 0% and about 35% relative humidity. On an opposite side, each dehumidifier 108 is configured to receive a decontaminated air stream 128 from scrubber 110 and discharge rehumidified air to rehumidified air stream 130 having a higher humidity. Rehumidified air stream 130 may have a higher humidity than the humidity of decontaminated air stream 128. For example, rehumidified air stream 130 may have a humidity that is selected to maintain a humidity of cabin 102 between about 5% and about 75% relative humidity.

Carbon dioxide removal system 106 includes liquid sorbent loop 136 configured to circulate liquid sorbent between scrubber 110 and stripper 112. For example, a pump 142 may pump unloaded liquid sorbent from stripper 112 into scrubber 110. Unloaded liquid sorbent may include unused liquid sorbent free of contaminants or regenerated liquid sorbent having a lower concentration of contaminants than the loaded liquid sorbent. In some examples, the unloaded liquid sorbent may be cooled by a cooler 144 prior to entry into carbon dioxide scrubber 110. In some examples, the loaded liquid sorbent may be preheated by a heat exchanger 138 and/or heater 140 prior to entry into stripper 112. A liquid sorbent storage 146 may store liquid sorbent, such as in a relatively cool state.

Scrubber 110 is configured to absorb carbon dioxide from dehumidified air stream 126 into the liquid sorbent and discharge decontaminated air stream 128 to membrane dehumidifiers 108. On a gas phase side, scrubber 110 is configured to receive dehumidified air from dehumidified air stream 126 that includes carbon dioxide from cabin 102. Scrubber 110 includes one or more separation membranes, each configured to flow (e.g., provide or direct flow of) dehumidified air from dehumidified air stream 126 on a gas phase side (e.g., a tube side) of the respective membrane and flow the liquid sorbent on a liquid phase side (e.g., a shell side) of the membrane. Contaminants may pass through the membrane due to a concentration gradient between the dehumidified air and the liquid sorbent and become absorbed by the liquid sorbent, while the liquid sorbent may not substantially flow through the membrane. As a result, decontaminated air from decontaminated air stream 128 discharged from scrubber 110 may have a lower concentration of carbon dioxide than dehumidified air from dehumidified air stream 126 received by scrubber 110. For example, decontaminated air stream 128 may have a concentration of carbon dioxide that is about 25% to about 99% less than a concentration of carbon dioxide in dehumidified air stream 126. Scrubber 110 is configured to discharge decontaminated air stream 128 to humidity management system 104. On a liquid phase side, scrubber 110 is configured to receive unloaded liquid sorbent, such as from liquid sorbent storage 146. The unloaded second liquid sorbent may flow through scrubber 110 and absorb carbon dioxide and other gaseous contaminants from dehumidified air through the membrane(s) of scrubber 110. As a result, the loaded liquid sorbent discharged from scrubber 110 may have a higher concentration of carbon dioxide than the unloaded second liquid sorbent received by scrubber 110. Scrubber 110 may discharge the loaded second liquid sorbent containing the carbon dioxide to stripper 112.

Stripper 112 is configured to desorb the carbon dioxide from the liquid sorbent into contaminant stream 120. On a liquid phase side, carbon dioxide stripper 112 is configured to receive loaded liquid sorbent from scrubber 110 and desorb carbon dioxide from the loaded liquid sorbent. Stripper 112 includes one or more membranes, each configured to flow the loaded liquid sorbent on one side (e.g., a shell side) of the membrane and contaminated air to contaminant stream 120 on an opposite side (e.g., a tube side) of the membrane. Carbon dioxide may flow across fibers of the membrane due to a concentration gradient, while the liquid sorbent may not substantially flow across the fibers of the membrane. As a result, unloaded liquid sorbent discharged from stripper 112 may have a lower concentration of carbon dioxide than the loaded liquid sorbent received by stripper 112. On a gas phase side, stripper 112 is configured to discharge the carbon dioxide in contaminant stream 148. Contaminant stream 148 may be continuously removed from stripper 112 to assist migration of the carbon dioxide from the loaded liquid sorbent into contaminant stream 148.

Scrubber 110, and/or stripper 112 may include one or more membrane separators configured to flow air on a first side and flow liquid sorbent on a second, opposite side. For example, a membrane separator may include a plurality of parallel membrane contactors. In some examples, a membrane contactor may include a cylindrical module filled with parallel or woven hollow porous fibers forming a hydrophobic porous membrane. For example, dimensions of these hollow fibers could be less than about 3 mm, and the pore dimension could be less than about 2 microns. The high surface area of the hollow fiber membrane contactors enables a high mass transfer of contaminant gases, such as carbon dioxide and water, into the respective liquid sorbent using a relatively small system volume and weight. The material of the hollow fibers can be selected such that the liquid sorbent does not wet the pores, and the trans-membrane pressure is kept sufficiently low to prevent pore penetration. As a result, the membrane contactor may ensure that the liquid sorbent and gas stream do not need further separation, such that contaminant removal system 100A may act in a gravity-independent way without the use of moving parts. Fiber materials may include, but are not limited to, hydrophobic materials such as polypropylene, polyvinylidene fluoride, polysulfone, polyimide, polytetrafluoroethylene (PTFE), and the like. In some examples, a coating may be applied to reduce liquid flow through the pores. Coatings that may be used include, but are not limited to, PTFE, a crosslinked siloxane, perfluorinated polymers, functionalized nanoparticles, and the like to prevent liquid flow through the pores. While described in FIG. 3 as flowing through a “tube” side, liquid sorbent flow can be either on the “tube” side or the “shell” side, while gas is flowed on the opposite side.

In the example of FIG. 3, contaminant removal system 100 may include one or more systems or components configured to further process contaminant stream 148. In some examples, contaminant removal system 100 includes a filter 150, a compressor 152, a condenser 154, and a water separator 156 configured to compress contaminant stream 148 and remove water from the compressed contaminant stream 148. For example, for carbon dioxide removed from contaminant removal system 100 to be stored or recycled, compressor 152, condenser 154, and water separator 156 may compress contaminant stream 148 to a high pressure and remove nearly all water from contaminant stream 148. In a life support application, a large amount of water may be present in cabin air stream 120. For example, the humidity in cabin air stream 120 may be much higher than that of carbon dioxide. Sabatier reactor 164 may be configured to generate one or more hydrocarbons using the removed carbon dioxide, and may require a water concentration of less than 2% to react hydrogen gas with carbon dioxide.

Filter 150 is configured to remove any leaked liquid sorbent and/or further filter clean contaminants from contaminant stream 148. Compressor 152 is configured to compress contaminant stream 148. A variety of compressors may be used for compressor 152 including, but not limited to, centrifugal compressors, positive displacement compressors, and the like. Condenser 154 may be configured to cool contaminant stream 148 and condense water from contaminant stream 148. For example, condenser 154 may be coupled to a refrigeration system or other cooling system that circulates a cooling medium to cool contaminant stream 148. A variety of condensers may be used for condenser 154 including, but not limited to, shell and tube heat exchangers, plate-fin, surface coolers, heat pipes, thermoelectric devices, cooling jackets, and the like. Water separator 156 may be configured to remove water from contaminant stream 148, discharge a dehumidified contaminant stream 158 to Sabatier reactor 164, and discharge water condensate stream 160 to water storage 162. A variety of water separators may be used for water separator 156 including, but not limited to, static phase separators, capillary phase separator, membrane phase separators, centrifugal/rotary separators, and the like.

Controller 114 (not shown) may be communicatively coupled to and configured to receive measurement signals from one or more sensor sets, and other process control components (not shown) of contaminant removal system 100, such as: control valves for cabin air stream 120, dehumidified air stream 126, decontaminated air stream 128, rehumidified air stream 130, contaminant stream 148, and inlets/outlets to heat exchanger 138, heater 140, liquid sorbent storage 146, and cooler 144; pumps 142; blower 124, compressor 152 (e.g., pumping speed); and the like.

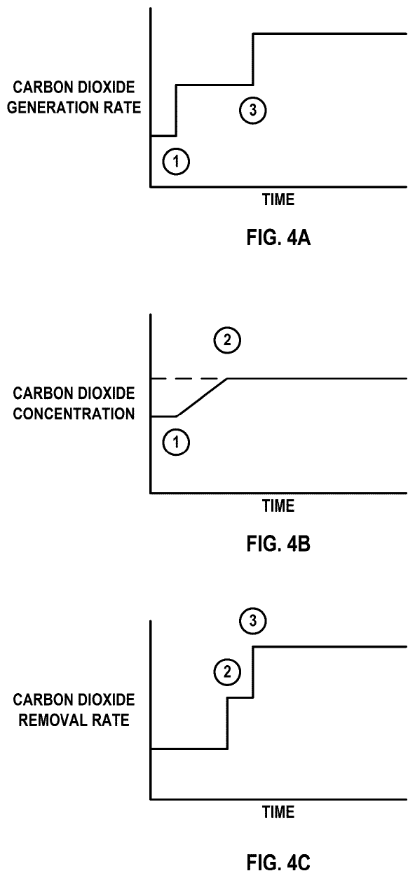

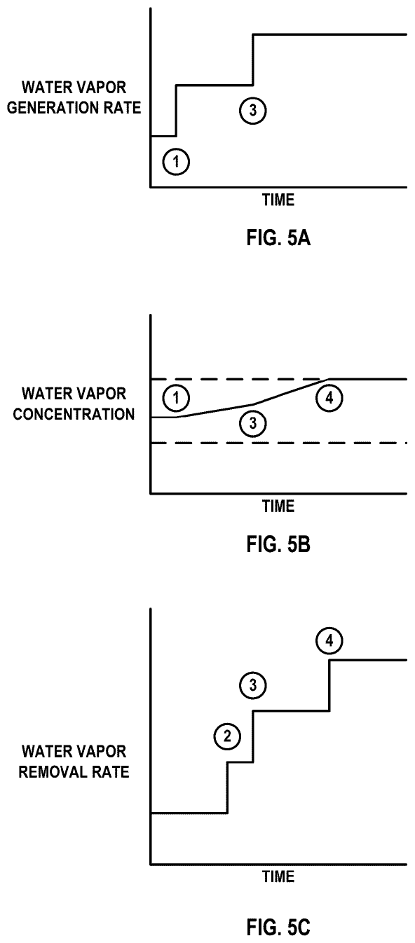

FIGS. 4A-4C are graphs illustrating removal of carbon dioxide from a cabin air stream, while FIG. 5A-5C are graphs illustrating removal of water vapor from the cabin air stream. At Time 1, a number of personnel in cabin 102, an activity level of personnel in cabin 102, or a level of operation of a system in cabin 102 may increase, resulting in an increase in a carbon dioxide generation rate (FIG. 4A) and an increase in water vapor generation rate (FIG. 5A). A carbon dioxide concentration in cabin 102 may increase until, at Time 2, the carbon dioxide concentration reaches a threshold (FIG. 4B). Correspondingly, a water vapor concentration in cabin 102 may also increase, though at a slower rate relative to the carbon dioxide concentration (FIG. 5B). In response to the carbon dioxide concentration reaching or approaching the threshold, controller 114 may operate carbon dioxide removal system 106 to increase removal of carbon dioxide from the cabin air stream (FIG. 5C). To preserve a humidity of the cabin air stream, controller 114 may also operate humidity management system 104 to increase removal of water vapor from the cabin air stream and return the water vapor to the decontaminated air stream by selecting a capacity corresponding to the desired water vapor removal rate (FIG. 5C). For example, controller 114 may select a single membrane dehumidifier to achieve the desired water vapor removal rate.

At Time 3, a number of personnel in cabin 102 may further increase, resulting in a further increase in a carbon dioxide generation rate (FIG. 4A) and a further increase in water vapor generation rate (FIG. 5A). In response to the increase in the carbon dioxide generation rate, controller 114 may operate carbon dioxide removal system 106 to increase removal of carbon dioxide from the cabin air stream to maintain the carbon dioxide concentration at or below the threshold (FIG. 4C). Correspondingly, controller 114 may operate humidity removal system 104 to remove water from the cabin air stream and return the removed water to the decontaminated air stream (FIG. 5C). For example, controller 114 may select an additional membrane dehumidifier 108 in parallel or series to increase a capacity of humidity removal system 104.

A humidity in the cabin air stream may continue to increase until, at Time 4, the water vapor concentration reaches an upper threshold (FIG. 5B). In response to the water vapor concentration reaching the threshold, controller 114 may operate humidity management system 104 to further increase an amount of water vapor removed from the cabin air stream (FIG. 5C). Additionally or alternatively, scrubber 110 may absorb the water vapor to replenish water that may otherwise be desorbed from the liquid sorbent mixture. In this way, humidity management system 104 having a variable capacity may both manage a humidity within cabin 102 and a water concentration in carbon dioxide removal system 106.

By capturing humidity from cabin air prior to entry of cabin air from the cabin air stream into scrubber 110, a greater amount of water may be removed from the cabin air stream prior to being received by scrubber 110 and/or a reduced amount of power may be used to remove water from the cabin air stream by humidity management system 110. Each membrane dehumidifier 108 may operate with relatively low power (e.g., power to pump gases or liquids). Removing water prior to going through scrubber 110 may result in less excess water that is absorbed into the liquid sorbent and, corresponding, less water to be removed, such that a load of compressor 170 may be decreased. This water removal may allow for smaller sizing of scrubber 110 and/or stripper 112, and/or a smaller load on components of carbon dioxide removal system.

Example 1: A contaminant removal system includes a humidity management system configured to remove water vapor from a cabin air stream to produce a dehumidified air stream and add water vapor to a decontaminated air stream, wherein the humidity management system includes two or more membrane dehumidifiers; and a carbon dioxide removal system downstream of the humidity management system and configured to remove carbon dioxide from the dehumidified air stream using a liquid sorbent and discharge a decontaminated air stream.

Example 2: The contaminant removal system of example 1, wherein the carbon dioxide removal system comprises: a scrubber configured to absorb one or more contaminants from the dehumidified air stream into the liquid sorbent, wherein the one or more contaminants include carbon dioxide; and a stripper configured to desorb the one or more contaminants from the liquid sorbent.

Example 3: The contaminant removal system of any of examples 1 and 2, wherein each of the two or more membrane dehumidifiers comprises a hollow fiber membrane dehumidifier.

Example 4: The contaminant removal system of any of examples 1 through 3, further comprising a manifold system configured to selectively couple each of the two or more membrane dehumidifiers to receive the cabin air stream.

Example 5: The contaminant removal system of any of examples 1 through 4, wherein the two or more membrane dehumidifiers are fluidically coupled in parallel with respect to the cabin air stream.

Example 6: The contaminant removal system of any of examples 1 through 5, wherein the two or more membrane dehumidifiers are fluidically coupled in series with respect to the cabin air stream.

Example 7: The contaminant removal system of any of examples 1 through 6, wherein at least two of the two or more membrane dehumidifiers have a different capacity.

Example 8: The contaminant removal system of any of examples 1 through 7, wherein each of the two or more membrane dehumidifiers has a similar capacity.

Example 9: The contaminant removal system of any of examples 1 through 8, wherein a removal rate of water by the humidity management system is independent of a removal rate of carbon dioxide by the carbon dioxide removal system.

Example 10: The contaminant removal system of any of examples 1 through 9, further comprising a Sabatier reactor configured to generate one or more hydrocarbons using the removed carbon dioxide.

Example 11: A method for removing contaminants from an environment includes removing, by a humidity management system, water vapor from a cabin air stream to produce a dehumidified air stream, wherein the humidity management system includes two or more membrane dehumidifiers; adding, by the humidity management system, water vapor to a decontaminated air stream; removing, by a carbon dioxide removal system, carbon dioxide from the dehumidified air stream using a liquid sorbent; and discharging, by the carbon dioxide removal system, a decontaminated air stream.

Example 12: The method of example 11, wherein removing carbon dioxide comprises: absorbing, by a scrubber, one or more contaminants from the dehumidified air stream into the liquid sorbent, wherein the one or more contaminants include carbon dioxide; and desorbing, by a stripper, the one or more contaminants from the liquid sorbent.

Example 13: The method of any of examples 11 and 12, wherein each of the two or more membrane dehumidifiers comprises a hollow fiber membrane dehumidifier.

Example 14: The method of any of examples 11 through 13, further comprising selectively coupling, by a manifold system, each of the two or more membrane dehumidifiers to receive the cabin air stream.

Example 15: The method of any of examples 11 through 14, wherein the two or more membrane dehumidifiers are fluidically coupled in parallel with respect to the cabin air stream.

Example 16: The method of any of examples 11 through 15, wherein the two or more membrane dehumidifiers are fluidically coupled in series with respect to the cabin air stream.

Example 17: The method of any of examples 11 through 16, wherein at least two of the two or more membrane dehumidifiers have a different capacity.

Example 18: The method of any of examples 11 through 17, wherein each of the two or more membrane dehumidifiers has a similar capacity.

Example 19: The method of any of examples 11 through 18, wherein a removal rate of water by the humidity management system is independent of a removal rate of carbon dioxide by the carbon dioxide removal system.

Example 20: The method of any of examples 11 through 19, further comprising generating, by a Sabatier reactor, one or more hydrocarbons from the removed carbon dioxide.

Various examples have been described. These and other examples are within the scope of the following claims.

What is claimed is:

1. A contaminant removal system, comprising:
a humidity management system configured to remove water vapor from a cabin air stream to produce a dehumidified air stream and add water vapor to a decontaminated air stream, wherein the humidity management system includes two or more membrane dehumidifiers; and
a carbon dioxide removal system downstream of the humidity management system and configured to remove carbon dioxide from the dehumidified air stream using a liquid sorbent and discharge a decontaminated air stream.

2. The contaminant removal system of claim 1, wherein the carbon dioxide removal system comprises:
a scrubber configured to absorb one or more contaminants from the dehumidified air stream into the liquid sorbent, wherein the one or more contaminants include carbon dioxide; and
a stripper configured to desorb the one or more contaminants from the liquid sorbent.

3. The contaminant removal system of claim 1, wherein each of the two or more membrane dehumidifiers comprises a hollow fiber membrane dehumidifier.

4. The contaminant removal system of claim 1, further comprising a manifold system configured to selectively couple each of the two or more membrane dehumidifiers to receive the cabin air stream.

5. The contaminant removal system of claim 1, wherein the two or more membrane dehumidifiers are fluidically coupled in parallel with respect to the cabin air stream.

6. The contaminant removal system of claim 1, wherein the two or more membrane dehumidifiers are fluidically coupled in series with respect to the cabin air stream.

7. The contaminant removal system of claim 1, wherein at least two of the two or more membrane dehumidifiers have a different capacity.

8. The contaminant removal system of claim 1, wherein each of the two or more membrane dehumidifiers has a similar capacity.

9. The contaminant removal system of claim 1, wherein a removal rate of water by the humidity management system is independent of a removal rate of carbon dioxide by the carbon dioxide removal system.

10. The contaminant removal system of claim 1, further comprising a Sabatier reactor configured to generate one or more hydrocarbons using the removed carbon dioxide.

11. A method for removing contaminants from an environment, comprising:
removing, by a humidity management system, water vapor from a cabin air stream to produce a dehumidified air stream, wherein the humidity management system includes two or more membrane dehumidifiers;
adding, by the humidity management system, water vapor to a decontaminated air stream;
removing, by a carbon dioxide removal system, carbon dioxide from the dehumidified air stream using a liquid sorbent; and
discharging, by the carbon dioxide removal system, a decontaminated air stream.

12. The method of claim 11, wherein removing carbon dioxide comprises:
absorbing, by a scrubber, one or more contaminants from the dehumidified air stream into the liquid sorbent, wherein the one or more contaminants include carbon dioxide; and
desorbing, by a stripper, the one or more contaminants from the liquid sorbent.

13. The method of claim 11, wherein each of the two or more membrane dehumidifiers comprises a hollow fiber membrane dehumidifier.

14. The method of claim 11, further comprising selectively coupling, by a manifold system, each of the two or more membrane dehumidifiers to receive the cabin air stream.

15. The method of claim 11, wherein the two or more membrane dehumidifiers are fluidically coupled in parallel with respect to the cabin air stream.

16. The method of claim 11, wherein the two or more membrane dehumidifiers are fluidically coupled in series with respect to the cabin air stream.

17. The method of claim 11, wherein at least two of the two or more membrane dehumidifiers have a different capacity.

18. The method of claim 11, wherein each of the two or more membrane dehumidifiers has a similar capacity.

19. The method of claim 11, wherein a removal rate of water by the humidity management system is independent of a removal rate of carbon dioxide by the carbon dioxide removal system.

20. The method of claim 11, further comprising generating, by a Sabatier reactor, one or more hydrocarbons from the removed carbon dioxide.
